(19) United States
(12) Patent Application Publication
(10) Pub. No.: US 2025/0152215 A1
(43) Pub. Date: May 15, 2025

(54) Orthopedic Implant In The Form Of A Plate To Be Fixed Between Two Bone Parts

(71) Applicant: Stryker European Operations Holdings LLC., Portage, MI (US)

(72) Inventors: Bernard Prandi, Rennes (FR); Keith Wapner, Philadelphia, PA (US); Charles P. Wapner, Media, PA (US); Peter W. Wapner, Media, PA (US)

(21) Appl. No.: 19/025,037

(22) Filed: Jan. 16, 2025

Related U.S. Application Data
(63) Continuation of application No. 17/990,936, filed on Nov. 21, 2022.
Application No. 17/990,936 is a continuation of application No. 16/429,834, filed on Jun. 3, 2019, now Pat. No. 11,534,212.
Application No. 16/429,834 is a continuation of application No. 15/130,147, filed on Apr. 15, 2016, now Pat. No. 10,349,988.
Application No. 15/130,147 is a continuation of application No. 14/734,676, filed on Jun. 9, 2015, now Pat. No. 9,333,013.
Application No. 14/734,676 is a continuation of application No. 14/041,706, filed on Sep. 30, 2013, now Pat. No. 9,078,713.
Application No. 14/041,706 is a continuation of application No. 12/918,071, filed on Oct. 29, 2010, now Pat. No. 8,556,946.

(30) Foreign Application Priority Data
Oct. 2, 2008 (FR) 0856694

(51) Int. Cl. A61B 17/80; A61B 17/68; A61B 17/84

(52) U.S. Cl. CPC A61B 17/8014; A61B 17/8004; A61B 17/8052; A61B 17/8057; A61B 17/8061; A61B 17/808; A61B 17/809; A61B 17/846; A61B 2017/681

(57) ABSTRACT

The invention relates to a plate fixed between two bone parts by way of screws engaged in holes formed in the thickness of said plate. The plate comprises an angled member or rib which is inclined according to an angle of between about 30° and 60° in relation to the plane defined by the plate. The angled member or rib has a hole for engaging a screw and is located in the central part of the width, over a determined part of the length of the plate, so that the screw brings the two bone parts into a compressive position.

### CROSS-REFERENCE TO RELATED APPLICATIONS

This application is a continuation of U.S. application Ser. No. 17/990,936, filed Nov. 21, 2022, which is a continuation of U.S. application Ser. No. 16/429,834, filed Jun. 3, 2019, now U.S. Pat. No. 11,534,212, which is a continuation of U.S. application Ser. No. 15/130,147, filed Apr. 15, 2016, now U.S. Pat. No. 10,349,988, which is a continuation of U.S. application Ser. No. 14/734,676, filed Jun. 9, 2015 and now U.S. Pat. No. 9,333,013, which is a continuation of U.S. application Ser. No. 14/041,706, filed Sep. 30, 2013 and now U.S. Pat. No. 9,078,713, which is a continuation of U.S. application Ser. No. 12/918,071, filed Oct. 29, 2010 and now U.S. Pat. No. 8,556,946, which is a national phase entry under 35 U.S.C. § 371 of International Application No. PCT/FR2009/051879, filed Oct. 2, 2009, published in French, which claims priority from French Patent Application No. 0856694, filed Oct. 2, 2008, all of which are incorporated herein by reference in their entireties.

### BACKGROUND OF THE INVENTION

The invention relates to the technical field of orthopedic implants.

More particularly, the invention relates to a plate for arthrodesis or osteosynthesis adapted to be fixed between two bone parts.

In a manner known to one having ordinary skill in the art, this type of plate generally has holes for engaging screws, allowing arthrodesis between two bones or osteosynthesis between two bone fragments. This is, for example, the case for bones of the hand or foot, without however excluding other applications, particularly in the field of the spine. Depending on the pathology to be treated, these plates can have a general rectilinear or other geometric shapes.

From this state of the art, one of the objects the invention proposes to attain is to improve, in a sure and efficient manner, compression in a precise direction between the bone parts subjected to the plate.

To attain the given object to enhance the compression between the two relative bone parts, according to the invention, the plate has a formation that orients at least one screw at an angle with respect to a plane defined by the plate, the angle being between about 30° and 60°.

According to an advantageous embodiment, the formation is a tab that is angled according to an angle between 30° and 60°, and having a hole for engaging the screw. The angled tab results from a cut out and a deformation of a portion of the plate.

In another embodiment, the formation is a hole angled at an angle between 30° and 60° for engaging the screw.

Considering the problem to be solved, the formation is located on a determined portion of the length of the plate so that the screw ensures the compression of the two bone parts.

### BRIEF DESCRIPTION OF THE DRAWINGS

The invention is described hereinafter in more detail, with reference to the attached drawings in which:

FIG. 1 is a perspective view of an embodiment of the plate;

FIG. 2 is a side view of the plate;

FIGS. 3 and 4 are perspective views showing the mounting of the plate between two bone parts and their orientation by means of the plate according to the invention, the bone parts being shown schematically.

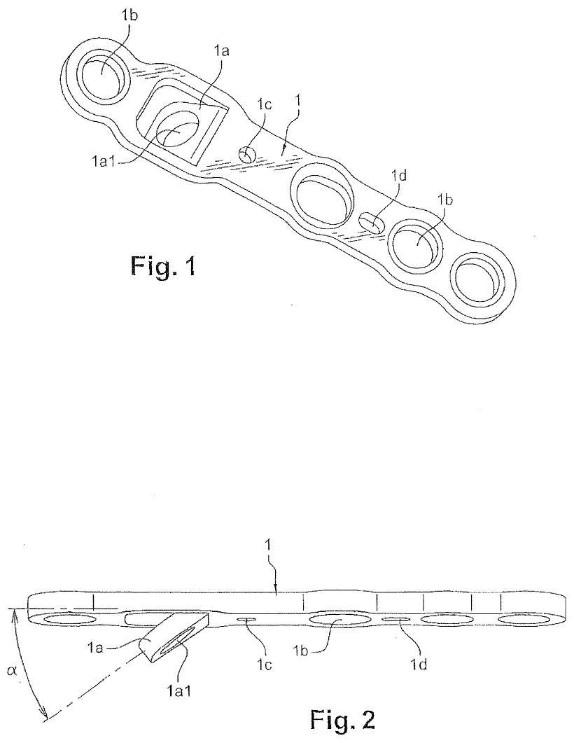

### DETAILED DESCRIPTION

According to the invention, the plate 1 has at least one formation 1a adapted to enable the positioning of at least one screw 2, at an angle α of between 30° and 60° with respect to a plane of the plate (FIG. 2).

In one embodiment, the formation 1a is an angled tab cut out and deformed from the plate. For example, the deformation is made with a cutting-punching operation. This angled tab has a hole 1a1 for screw 2. The angled tab 1a is positioned along the length of the plate so that after the screw 2 is fitted to it, the screw ensures the compression together of the two bone parts, as indicated below in the description.

In another embodiment, to allow for an angular orientation of the screw 2 according to an angle between about 30° and 60°, the formation 1a can be formed as an angled hole. It must be noted that the tab 1a enables adaptation of the angle as a function of the pathology to be treated, given that it is possible to deform this tab at will. In other words, the angle can be adjusted over a few degrees directly by the surgeon in the operating room, using an appropriate tool.

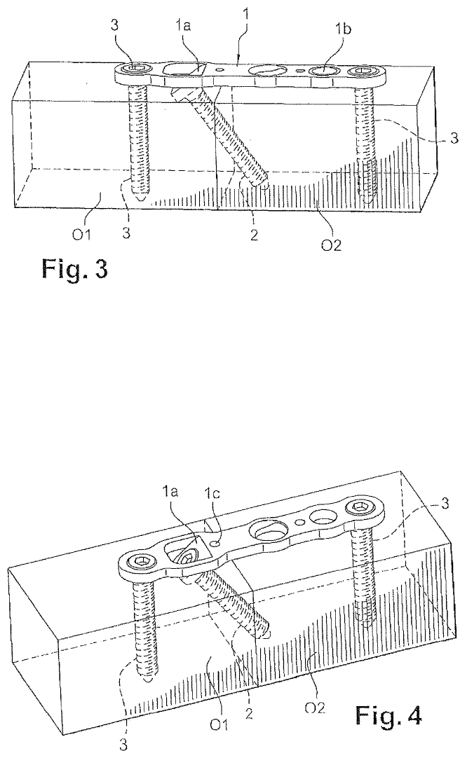

With reference to FIGS. 3 and 4 that show the positioning of the plate 1 between two bone parts O1 and O2:

Once the osteotomies have been carried out, a template of the plate, which does not have a guide formation, enables the position of the tab to be determined.

After determining the position of the tab, the surgeon makes a corresponding recess with the appropriate rasp.

Once the plate having the tab has been positioned, the surgeon sets one or two screws 3, on a side of the site of the osteosynthesis or the arthrodesis toward the tab. A temporary fastening pin can, possibly, be positioned in a complementary lug.

The screw 2 is then engaged in the hole 1a1 of the tab 1a to place the fracture in compression.

Once the compression has been done, the surgeon can screw one or several other additional fastening screws 3 and remove the temporary pin.

In a known manner, this plate 1 has smooth and/or threaded holes for the fastening screws 3 set in the bone parts O1 and O2 to engage in, as shown in FIGS. 3 and 4.

Similarly, the plate 1 can have at least one hole 1c for a pin for temporarily positioning the plate 1. Advantageously, the plate 1 can have a guide 1c for the insertion of a pin on the side of one of the bone parts O1 and another guide 1d for the insertion of another pin on the side of the other bone part O2.

Considering the effect of the desired compression, such as indicated above, the guide 1c is a circular hole whose diameter corresponds substantially to that of the pin, whereas the other guide 1d can be an elongated slot.

These provisions thus enable the bone to slide under the plate 1 as the screws are set, while ensuring compression along a precise direction, generally axially or parallel to the plate. The pins are of any known and appropriate type, and perfectly known to one having ordinary skill in the art.

The plate 1 can have several shapes, so that the holes 1a in particular can be aligned or arrayed, all or in part, according to the corners of a triangle or of a quadrilateral. These provisions, in triangle or in quadrilateral, of the screws, improve the stability of the mounting.

It must be noted also that the plate 1, no matter its shape, can be longitudinally bent so as to adapt to the curvature of the bone, consequently enabling the screws to form an angle between them.

The advantages are readily apparent from the description.

1. (canceled)

2. A bone plate system for fusing a first bone portion and a second bone portion, said system comprising:
a bone plate defined by a main body extending generally longitudinally from a first end to a second end including:
an upper surface;
a lower surface;
the first end configured to extend over the first bone portion, the first end having a first bone screw hole;
the second end configured to extend over the second bone portion, the second end having a second bone screw hole; and
an angled third bone screw hole located along the main body between the first bone screw hole and the second bone screw hole, wherein the angled third bone screw hole is disposed at a non-perpendicular angle with respect to both the upper and lower surfaces;
a first fixation member configured to be inserted through the first bone screw hole and into the first bone portion;
a second fixation member configured to be inserted through said second bone screw hole and into the second bone portion; and
a third fixation member configured to be inserted through the third bone screw hole, into the first bone portion, and into the second bone portion such that a free end of said third fixation member, not attached to any portion of the bone plate, resides in the second bone portion, wherein a head of the third fixation member is located below the upper surface.

3. The system of claim 2, wherein the bone plate is contoured to anatomically fit bones in a human foot.

4. The system of claim 2, wherein the first bone portion and the second bone portion are separated by a metatarsophalangeal joint.

5. The system of claim 2, wherein the first bone portion and the second bone portion are separated by a tarsometatarsal joint.

6. The system of claim 2, wherein the third fixation member is configured to develop compression between the first and second bone portions when said third fixation member is tightened.

7. The system of claim 2, wherein free ends of the second and third fixation members are configured to reside adjacent each other within the second bone portion.

8. The system of claim 2, wherein the bone plate includes at least one pin hole adjacent the first bone screw hole, the pin hole configured to receive a temporary fixation member.

9. The system of claim 2, wherein the third bone screw hole extends through an angled member recessed below the upper surface.

10. The system of claim 9, wherein the angle member is situated below a guide slot formed through the plate.

11. A bone plate comprising a main body extending generally longitudinally from a first end to a second end, the main body comprising:
an upper surface;
a lower surface;
the first end configured to extend over a first bone portion, the first end having a first hole configured to receive a first fixation member;
the second end configured to extend over a second bone portion, the second end having a second hole configured to receive a second fixation member; and
an angled third hole located on the main body between the first hole and the second hole, said angled third hole disposed at a non-perpendicular angle with respect to both the upper and lower surfaces and configured to receive a third fixation member having a third head, such that the third head is located below the upper surface.

12. The bone plate of claim 11, wherein the bone plate is contoured to anatomically fit bones in a human foot.

13. The bone plate of claim 11, wherein the first bone portion and the second bone portion are separated by a metatarsophalangeal joint.

14. The bone plate of claim 11, wherein the first bone portion and the second bone portion are separated by a tarsometatarsal joint.

15. The bone plate of claim 11, wherein the third fixation member is configured to develop compression between the first and second bone portions when the third fixation member is tightened.

16. The bone plate of claim 11, wherein the bone plate includes at least one pin hole adjacent the first hole, the pin hole configured to receive a temporary fixation member.

17. The bone plate of claim 11, wherein the third hole extends through an angled member recessed below an upper surface of the bone plate.

18. The bone plate of claim 17, wherein the angled member is situated below a guide slot formed through the plate.

19. The bone plate of claim 11, wherein the bone plate is substantially planar.

20. A bone plate system comprising:
the bone plate of claim 11; and
a fixation member.

21. The bone plate of claim 11, further comprising an elongate slot.
